(19) United States
(12) Patent Application Publication
(10) Pub. No.: US 2011/0046562 A1
(43) Pub. Date: Feb. 24, 2011

(54) SPLIT SYRINGE DEVICE

(76) Inventor: JIN WANG, Claremont, CA (US)

Correspondence Address: JIN WANG, 1016 TREVECCA PLACE, CLAREMONT, CA, 91711, US

(21) Appl. No.: 12/859,221

(22) Filed: Aug. 18, 2010

Related U.S. Application Data
(60) Provisional application No. 61/234,711, filed on Aug. 18, 2009.

(51) Int. Cl. A61M 5/31

(52) U.S. Cl. 604/191

(57) ABSTRACT

A split syringe includes two or more separate syringes held together to become one full cylindrically shaped syringe assembly. The needles of the separate syringes may be held together by a larger outside needle. For example, two half-cylindrically shaped syringes may be joined together to become one full cylindrically shaped syringe assembly.

### CROSS-REFERENCE TO RELATED APPLICATIONS

This application claims the benefit of priority of U.S. provisional patent application No. 61/234,711, filed Aug. 18, 2009, which is herein incorporated by reference in its entirety.

### BACKGROUND OF THE INVENTION

The present invention relates to syringes and, more particularly, to a split syringe assembly that may permit filling of multiple syringes with a single action.

Liquid medications are often supplied in vials having a septum sealing the vial. To withdraw the medicine from the vial, a needle of a syringe is inserted through the septum and the medicine is taken up into the syringe. Some medicine (because of the nature of the medicine or due to insurance company requirements) may be withdrawn from a vial only one time. If the dose to be withdrawn is less than the amount of medicine in the vial, the medicine left behind is wasted. If the vial is a multi-use vial (allowing multiple insertions of syringes), repeated insertions may present safety issues (due to an increased risk of needle injury) and contamination issues (due to inserting multiple needles into the vial). Finally, multiple vial insertions, syringe plunger extensions and syringe withdrawals require repeated and numerous actions on the medical professional.

As can be seen, there is a need for a syringe device that may permit multiple syringes to be filled with a single action and a single needle insertion.

### SUMMARY OF THE INVENTION

In one aspect of the present invention, a split syringe assembly comprises a first syringe having a first plunger, a first syringe tube segment and a first needle; and a second syringe having a second plunger, a second syringe tube segment and a second needle; and an external needle fitting over the first needle and the second needle, wherein the first syringe and the second syringe fit together to form the split syringe assembly with a single cylindrically shaped tube segment.

In another aspect of the present invention, a split syringe assembly comprises a first half cylindrical syringe having a first half cylindrical plunger, a first half cylindrical syringe tube segment, a first needle, and a first half cylindrical syringe base fluidly connecting an interior of the first needle with the first half cylindrical syringe tube segment; a second half cylindrical syringe having a second half cylindrical plunger, a second half cylindrical syringe tube segment, a second needle, and a second half cylindrical syringe base fluidly connecting an interior of the second needle with the second half cylindrical syringe tube segment; and an external needle fitting over and holding together the first half cylindrical syringe base and the second half cylindrical syringe base, wherein the first half cylindrical syringe and the second half cylindrical syringe fit together to form the split syringe assembly with a single cylindrically shaped tube segment.

These and other features, aspects and advantages of the present invention will become better understood with reference to the following drawings, description and claims.

### BRIEF DESCRIPTION OF THE DRAWINGS

FIG. 1 is a perspective view of a split syringe according to an embodiment of the present invention;

FIG. 2 is a partially exploded view of the split syringe of FIG. 1;

FIG. 3 is a partially exploded view of the split syringe of FIG. 1;

FIG. 4 is an exploded view of the split syringe of FIG. 1 showing the separation of the split syringes;

FIG. 5 is a cross-sectional view taken along line 5-5 of FIG. 1; and

FIG. 6 is a cross-sectional view of a split syringe according to an alternate embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides a split syringe having two or more separate syringes held together to become one full cylindrically shaped syringe assembly. The needles of the separate syringes may be held together by a larger outside needle. For example, two half-cylindrically shaped syringes may be joined together to become one full cylindrically shaped syringe assembly.

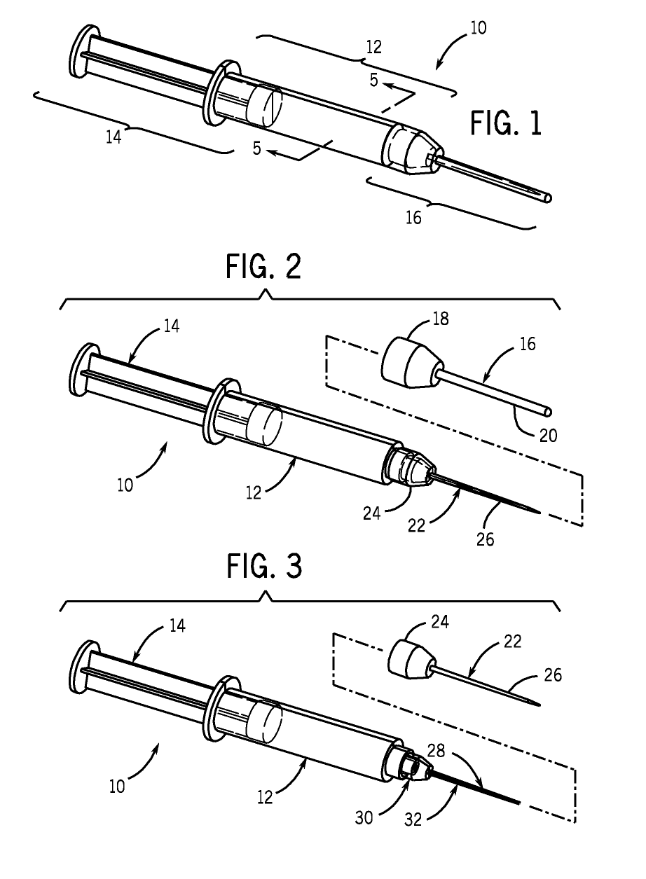

Referring to FIGS. 1 through 5, a split syringe assembly 10 may include a tube segment 12 and a plunger segment 14. A protective cap assembly 16 may cover a split syringe needle assembly 22. The protective cap assembly 16 may include a base 18 and a shroud 20. The split syringe needle assembly 22 may include a large needle 26 and a large needle base 24. The base 18 may fit over the large needle base 24 and the shroud 20 may fit over the large needle 26 when the protective cap assembly 16 is in position over the split syringe needle assembly 22.

Removal of the protective cap assembly 16 may reveal the split syringe needle assembly 22, as shown in FIG. 2. Removal of the split syringe needle assembly 22 may reveal two or more separate syringe needle assemblies 32, each of which may include a separate syringe needle 28 and a separate syringe base 30, as shown in FIG. 3.

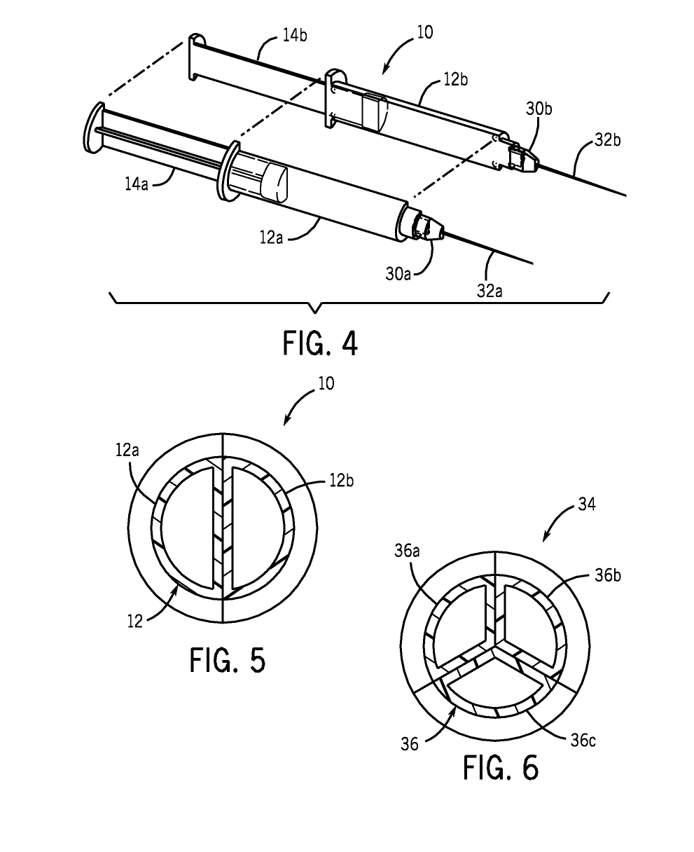

Referring to FIG. 4, with the split syringe needle assembly 22 removed, the split syringe 10 may be separated into multiple tube segments. For example, the split syringe 10 may be separated into a first half cylindrical tube segment 12a and a second half cylindrical tube segment 12b. Similarly, the needle base 24 may be separated into multiple needle bases. For example, as shown in FIG. 4, the needle base 24 may be separated into a first needle base 30a, connected to the first half cylindrical tube segment 12a, and a second needle base 30b, connected to the second half cylindrical tube segment 12b. Finally, the plunger assembly 14, when the split syringe needle assembly is separated, may be separated into plunger segments. For example, the plunger assembly 14 may be separated into a first plunger 14a, adapted to push fluid from or pull fluid into the first half cylindrical tube segment 12a, and a second plunger 14b, adapted to push fluid from or pull fluid into the second half cylindrical tube segment 12b. In other words, the split syringe assembly 10 may be comprised of multiple smaller syringes whose needles may be combined into a single larger needed (e.g., large needle 26).

While FIGS. 1-5 describe a split syringe assembly 10 that may be comprised of two half cylindrical syringes, any number of syringes may be combined to form the split syringe assembly 10. For example, as shown in FIG. 6, a split syringe assembly 34 may include a tube segment 36 that includes a first tube 36a, a second tube 36b and a third tube 36c, wherein the tubes 36a, 36b, 36c may be arranged to form the tube segment 36 in a cylindrical shape.

The split syringe assembly 10 may be used in a variety of ways. First, the split syringe assembly 10 may be used to draw two doses of a medication from a vial with a single injection. The large needle 26 may be injected into the vial (not shown) and the medicine may be drawn into the two half cylindrical syringes simultaneously with a single pull on the plunger assembly 14. Once the medicine is drawn, the large needle 26 may be removed from the vial and the needle may be removed and discarded from the separate syringe bases 30. The two half cylindrical syringes may be separated from each other and used for two different patients. This may not only saves wasted medicine from the single-use vial, but may also increase user safety by requiring only one needle stick into the vial and one pull of the plunger to create two doses of medicine.

The split syringe may also be used to inject two syringe volumes of medicine (or two different medicines) into an injection port of an intravenous line. A user may take up one medicine in one half syringe and a second medicine (or an additional volume of the same medicine) in the other half syringe. The large needle 26 may be placed on the split syringe 10 and the medicines in each half syringe may be administered either sequentially or simultaneously. This may result in two medicines being administered intravenously with only one needle stick into the injection port of the intravenous line.

The split syringe assembly may be made of materials and by methods known in the art. For example, the needle may be attached to the syringe body (i.e., the large needle 26 to the assembled split syringe bases 30) by a friction fit or by a conventional Luer lock. The large needle 26 may be, for example, from about 14 gauge to about 18 gauge. The separate needles 28 may be, for example, from about 22 gauge to about 24 gauge. The two half cylindrical syringes of the split syringe 10 may be aligned through the use of interlocking studs on each of the two half cylindrical syringes. For example, interlocking studs may be disposed on each distal lateral edge of the rectangular sides of the half cylindrically shaped syringe barrels. The two half cylindrical syringes may, for example, each be adapted to hold from about 1 cc to about 10 cc of fluid.

It should be understood, of course, that the foregoing relates to exemplary embodiments of the invention and that modifications may be made without departing from the spirit and scope of the invention as set forth in the following claims.

What is claimed is:

1. A split syringe assembly comprising:
a first syringe having a first plunger, a first syringe tube segment and a first needle; and
a second syringe having a second plunger, a second syringe tube segment and a second needle; and
an external needle fitting over the first needle and the second needle, wherein
the first syringe and the second syringe fit together to form the split syringe assembly with a single cylindrically shaped tube segment.

2. The split syringe assembly of claim 1, further comprising a third syringe having a third plunger, a third syringe tube segment and a third needle, wherein the external needle fits over the first needle, the second needle and the third needle and the first syringe, the second syringe and the third syringe fit together to form the split syringe assembly with a single cylindrically shaped tube segment.

3. The split syringe assembly of claim 1, wherein the first plunger and the second plunger fit together to form the split syringe assembly with a single cylindrically shaped plunger assembly.

4. The split syringe assembly of claim 1, further comprising:
a first syringe base fluidly connecting an interior of the first needle with the first syringe tube segment; and
a second syringe base fluidly connecting an interior of the second needle with the second syringe tube segment.

5. The split syringe assembly of claim 4, wherein the first syringe base and the second syringe base fit together to form the split syringe assembly with a single cylindrically shaped syringe base assembly.

6. The split syringe assembly of claim 5, wherein the external needle attaches to the single cylindrically shaped syringe base assembly.

7. A split syringe assembly comprising:
a first half cylindrical syringe having a first half cylindrical plunger, a first half cylindrical syringe tube segment, a first needle, and a first half cylindrical syringe base fluidly connecting an interior of the first needle with the first half cylindrical syringe tube segment;
a second half cylindrical syringe having a second half cylindrical plunger, a second half cylindrical syringe tube segment, a second needle, and a second half cylindrical syringe base fluidly connecting an interior of the second needle with the second half cylindrical syringe tube segment; and
an external needle fitting over and holding together the first half cylindrical syringe base and the second half cylindrical syringe base, wherein
the first half cylindrical syringe and the second half cylindrical syringe fit together to form the split syringe assembly with a single cylindrically shaped tube segment.

8. The split syringe assembly of claim 7, wherein the first plunger and the second plunger mate together and are adapted to be pulled simultaneously to withdraw a liquid through the external needle and into both the first half cylindrical syringe and the second half cylindrical syringe simultaneously.

9. The split syringe assembly of claim 7, further comprising a needle cap fitting over the external needle.

10. The split syringe assembly of claim 7, wherein the first plunger and the second plunger mate together and are adapted to be pushed either sequentially or simultaneously to deliver a liquid out through the external needle.
